(19) United States
(12) Patent Application Publication
(10) Pub. No.: US 2022/0040334 A1
(43) Pub. Date: Feb. 10, 2022

(54) HIGH-THROUGHPUT SCREENING METHODS OF SENESCENCE-ANTAGONIZING SUBSTANCES AND SYSTEMS THEREOF

(71) Applicant: MDI Biological Laboratory, Salisbury Cove, ME (US)

(72) Inventors: Roland Schmitt, Salisbury Cove, ME (US); Anette Melk, Salisbury Cove, ME (US); Vera Wulfmeyer, Salisbury Cove, ME (US); Maxine Swallow, Salisbury Cove, ME (US)

(73) Assignee: MDI Biological Laboratory, Salisbury Cove, ME (US)

(21) Appl. No.: 17/391,239

(22) Filed: Aug. 2, 2021

Related U.S. Application Data
(60) Provisional application No. 63/061,446, filed on Aug. 5, 2020.

(51) Int. Cl. A61K 49/00

(52) U.S. Cl. CPC A61K 49/0008

(57) ABSTRACT

A method and system for screening candidate substances or compositions thereof, where mammalian cells containing a stain or detectable marker are introduced into the yolk sac of a fertilized egg, embryo, or larvae of a transparent and/or translucent fish model. The method and system may also be employed to identify detectable marker genes, marker proteins, or marker metabolites in the transplanted mammalian cells, where the marker may indicate, e.g., cellular senescence, non-senescence, excessive or abnormal proliferation, damaged DNA. Methods of treating a subject suffering from a disease, disorder, or condition resulting from cellular senescence, excessive or abnormal proliferation, or damaged DNA using the candidate substances or compositions thereof are provided.

### RELATED APPLICATIONS

This application claims the priority of U.S. Provisional Patent Application No. 63/061,446, filed Aug. 5, 2020, to “HIGH-THROUGHPUT SCREENING METHODS OF SENESCENCE-ANTAGONIZING SUBSTANCES AND SYSTEMS THEREOF,” which is incorporated herein by reference in its entirety for all purposes. respectively. The government has certain rights in the invention.

### STATEMENT OF RIGHTS TO INVENTIONS MADE UNDER FEDERALLY SPONSORED RESEARCH

This invention was made with government support under Grant Nos. P20GM104318 and P20GM103423 awarded by the National Institute of General Medical Sciences: Centers of Biomedical Research Excellence (COBRE) and IDeA Networks of Biomedical Research Excellence (INBRE),

### BACKGROUND OF THE INVENTION

Cellular senescence is a basic mechanism present in most organisms. While cellular senescence has a physiological function during development it has also been identified as a major driving force of aging and of age-related diseases. Senescent cells are metabolically active and impact their environment through the Senescence-Associated Secretory Phenotype (SASP). Cellular senescence relates to stable cell cycle arrest. A senescent cell does not divide, avoids apoptosis, and secretes factors, such as but not limited to, inflammatory cytokines, immune modulators, growth factors, and proteases, that when in the cell present the Senescence-Associated Secretory Phenotype (SASP). The potential therapeutic effects of antagonizing the SASP or ablating senescent cells—a concept referred to as senolysis—has become an important field with high translational potential. Senolysis is thought to potentially launch novel therapeutic avenues for common age-related diseases.

Senescence in cells is linked to the aging process and age-related diseases. Aging and age-related diseases have an increase in senescent cells. Ablation of senescent cells has been shown to have some therapeutic effects in mouse models. The current ‘gold-standard’ which is practiced in laboratories globally is a two-dimensional cell culture of mammalian cells and the subsequent compound testing in mice, which involves mice tissue fixing, cutting, staining, and observing under the microscope. However, for preclinical senescence research and drug testing on the multicellular organismal level, the method of initially testing by in vitro cell culture followed by mouse models in vivo is impeded by the inability to detect promising in vitro effects in vivo. This lack in transferability between the two systems is very likely due to the fact that the complex in vivo macro- and micro-environment such as multicellularity, organismal metabolism, paracrine functions, and motile cell-cell interactions cannot be resembled by cell culture experiments. Moreover, mouse studies are further limited by costs, breeding time, and ethics. The workflow of extracting, preparing, and analyzing murine tissues is cumbersome.

Preclinical pipeline drug discovery is costly and time-consuming, which restricts the number of compounds that can be tested. There are presently no in vivo systems to test senescence-directed cellular strategies available for preclinical application. Therefore, there is a need in the art to design a robust method of screening target cells of higher mammalian organisms (including, for example, human, murine, and others) in a rather simple, but live organism in vivo.

### SUMMARY OF THE INVENTION

As described below, the present invention features senolytic/senotherapeutic drug screening methods and systems. Additionally, this method can also be useful in determining cellular mechanisms of senescence and the senescence-associated secretory phenotype (SASP). The methods and systems of the invention offer a step in between these two methods allowing compound testing in a high-throughput fashion using an in vivo model while at the same time being significantly more cost- and time effective. In contrast to mice, zebrafish embryos of the invention are translucent and the fate of xenografted senescent cells can be directly followed over time by fluorescence microscopy.

In one aspect, a method of analyzing cells may be provided, comprising: staining cells, such as mammalian cells, (e.g., non-senescent, senescent, epithelial cells, endothelial cells, primary tubular epithelial cells (PTECs), human umbilical vein endothelial cells (HUVECs)) with detectable markers or stains, such as but not limited to, detectable in vivo cell labels or live cell labeling reagents (e.g., fluorescent cell labels, e.g., eFluor and NucBlue stains); transgenic expression of fluorescent proteins (e.g., EGFP, dsRed); stains (e.g., γH2AX, Ki-67, SA-β-gal, DAPI), and the like; introducing one or more of the stained cells into a yolk sac of a fish (e.g., transparent, translucent, zebrafish, medaka), where the introduction may be by injection; incubating the fish in a medium; and analyzing the fish. The introducing step may comprise injecting the stained cells at least 1 day (e.g., 2 days, 3 days) post fertilization, or less than or equal to 20 days (e.g., 16 days, 12 days, 10 days, 8 days, 6 days, 4 days). The incubating step may occur at a temperature that is conducive to culturing both the fish and the mammalian cells introduced therein (e.g., 34° C.).

A further aspect of the analyzing step may comprise observing the fish, including the mammalian cells injected in the fish. Examining the fish may also be observed under a microscope by tracking the stained cells or marker in the fish. Another aspect may be directed to the analyzing step which comprises imaging the fish and its contents, including the transplanted mammalian cells. Yet a further aspect provides administering a candidate substance to the fish, where administering occurs by adding the candidate substance to the medium or directly injected into the fish or mammalian cells transplanted in the fish. The stained mammalian cells (e.g., human, murine) that may be transplanted into the transparent or translucent fish (e.g., zebrafish, medaka) may be senescent cells and/or non-senescent cells. In those aspects where the method provides for the method of administering a candidate substance, the candidate substances may be senotherapeutics, geroprotectors, Senescence Associated Secretory Phenotype (SASP) inhibitors, senolytics, or senomorphics. Some aspects of the method comprises analyzing the fish for any one or more of: a reversal of senescent cells to non-senescent cells, prevention of progression of non-senescent cells to senescent cells, senescent cell death, suppression of senescent phenotypes without cell killing, toxicity to proliferating cells, toxicity to senescent cells, suppression of cell senescence, increase in cell senescence, and increase in proliferation. The analyzing step of the method described here comprises quantifying senescence. In one aspect, quantifying senescence may be calculated by dividing total positive SA-β-Gal-stained area by the number of nuclei in DAPI-stained area.

Another aspect of the invention provides a method of screening for a candidate substance, comprising: staining cells (e.g., non-senescent, senescent, e.g., PTECs, HUVECs) with a detectable marker or stain, such as but not limited to, detectable in vivo cell label or live cell labeling reagents (e.g., fluorescent cell labels, e.g., eFluor and NucBlue stain); transgenic expression of fluorescent proteins (e.g. EGFP, dsRed); stain (e.g., γH2AX, Ki-67, SA-β-gal, DAPI), or the like; introducing (e.g., by injecting) one or more of the stained cells into a yolk sac of a fish (e.g., transparent/translucent-zebrafish, medaka); incubating the fish in a medium; administering a candidate substance (e.g., in the medium, directly injecting into the fish); and analyzing the fish after administration of the candidate substance. The candidate substance may be selected from: senotherapeutics, geroprotectors, Senescence Associated Secretory Phenotype (SASP) inhibitors, senolytics, and senomorphics, where the analysis of fish reveals any one or more of: a reversal of senescent cells to non-senescent cells, prevention of progression of non-senescent cells to senescent cells, senescent cell death, suppression of senescent phenotypes without cell killing, toxicity to proliferating cells, toxicity to senescent cells, suppression of cell senescence, increase in cell senescence, and increase in proliferation. The analyzing step may also comprise quantifying senescence, by staining the mammalian cells for cellular senescence and nuclei, using, for example, SA-β-Gal and DAPI. Quantification of senescence may be calculated by dividing total positive SA-β-Gal-stained area (i.e., marker for cellular senescence) by the number of nuclei in DAPI-stained area (i.e., marker for nuclei).

The invention provides methods and systems for screening potential senescence-antagonizing drugs in aging or age-related diseases, disorders, or conditions. Other features and advantages of the invention will be apparent from the detailed description and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a peptide, nucleic acid molecule, or small molecule or chemical compound.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, a 25% change, a 40% change, and a 50% or greater change in expression levels.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing,” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “detectable label” is meant any molecule or composition that when linked to a molecule of interest (e.g., analyte, detector reagent, analog, or binding partner) renders the latter detectable, via spectroscopic, photochemical, biochemical, immunochemical, electrical, optical, or chemical means. For example, useful labels include enzymes (for example, as commonly used in an ELISA), enzyme substrates, radioactive isotopes, magnetic beads, metallic beads, colloidal particles (e.g., colloidal gold particles), chemiluminescent or fluorescent dyes, electron-dense reagents, enzymes, biotin, digoxigenin, or haptens, and the like. Methods for labeling and guidance in the choice of labels appropriate for various purposes are discussed, e.g., in Green, M. R., Hughes, H., Sambrook, J., & McCallum, P. (2012). Molecular cloning: a laboratory manual. In Molecular cloning: a laboratory manual and Ausubel et al., Current Protocols in Molecular Biology. (1998). United States: John Wiley & Sons. The attachment of a compound (e.g., an antibody) to a label nay be through covalent bonds, adsorption processes, hydrophobic and/or electrostatic bonds, as in chelates and the like, or combinations of these bonds and interactions and/or may involve a linking group.

By “disease” is meant any condition or disorder that damages or interferes with the normal function of a cell, tissue, or organ. Non-limiting examples of diseases include aging or age-related disease, disorder, or conditions selected from neurodegenerating disease, myocardial infarction (i.e., heart attack), heart failure, atherosclerosis, hypertension, osteoarthritis, osteoporosis, sarcopenia, loss of bone marrow, cataract, multiple sclerosis, Sjögren's syndrome, Rheumatoid arthritis, degraded immune function, diabetes, Idiopathic pulmonary fibrosis, and age-related macular degeneration, cerebellar infarction, stroke, Alzheimer's disease, Parkinson's disease, Huntington's disease, tumorigenesis and malignant cancer development, inflammatory-related aging diseases, and disorders caused by the decline in testosterone, estrogen, growth hormone, IGF-I, or energy production.

The term “age-related disease” or “age-related disorder” refers to disorders or diseases where aging is a major risk factor, such as those categorized by: degenerative diseases, including neuron degenerating disease (e.g., Alzheimer's, Parkinson's, stroke), myocardial infarction, heart failure, atherosclerosis, hypertension, osteoarthritis, osteoporosis, sarcopenia, loss of bone marrow, rheumatoid arthritis, degraded immune function, diabetes, idiopathic pulmonary fibrosis, age-related macular degeneration; abnormal proliferative diseases (e.g., cancer); and disorders associated with a decrease in function, including a decrease in hormones (e.g., testosterone, estrogen, growth hormone, insulin-like growth factor I (IGF-I)), reduced energy production, and the like.

By “anti-aging effect” means phenotypes comprising increased mitochondrial biogenesis and function, reduced reactive oxygen species (ROS) levels, extended life span (e.g., cells (such as senescent cells), post-mitotic cells (such as neuron cells), tissues, organs, organisms), prevent ed age-related disorders (e.g., tumorigenesis, malignant progression of cancers, cerebellar infarction, and myocardial infarction).

“Senescence” as used herein, means a cell cycle-arrested state in mitotic cells. This may be induced by, for example, telomere dysfunction (e.g., altering the maintenance, function, or structure of telomeres), DNA damage (e.g., due to oxidative stress and/or oxidative), cellular response to environmental damage or disease or immune response or genetic alteration of cells, or oncogene activation. In mammalian cells (e.g., human, murine), senescent cells are known to be arrested at the G0 phase, i.e., a non-dividing phase not of the cell cycle. Senescence or senescent cells means that cells show no increase in number or an increase in cell death as observed under the microscope for 7 days after injection with senescent cells as compared with those injected with non-senescent cells, and exhibit β-galactosidase positive staining (e.g., senescence-associated beta-galactosidase (SA-β-gal)). Any mammalian cells that may undergo senescence are contemplated for use in the systems and methods described here, including, for example, the transparent fish model system and methods of analyzing and screening for senescence-antagonizing candidate substances. Specifically, any of these mammalian cells may be transplanted into the yolk sac of a transparent or translucent fish model system.

By “effective amount” or “therapeutically effective amount” of a candidate substance described here, as used herein, is that amount sufficient to effect beneficial or desired results, such as clinical results, and, as such, an “effective amount” depends upon the context in which it is being applied. In some embodiments, “effective amount” or “therapeutically effective amount” is meant the amount of a candidate substance required to ameliorate the symptoms of a disease relative to an untreated patient. In another embodiment, in the context of administering a candidate senescence-antagonizing substance, an effective amount of a candidate substance is, for example, an amount sufficient to achieve alleviation or amelioration or prevention or prophylaxis of one or more symptoms or conditions; diminishment of extent of disease, disorder, or condition; stabilized (i.e., not worsening) state of disease, disorder, or condition; preventing spread of disease, disorder, or condition; delay or slowing the progress of the disease, disorder, or condition; amelioration or palliation of the disease, disorder, or condition (e.g., senescence or symptoms of senescence), as compared to the response obtained without administration of the candidate substance. The effective amount of active candidate substances including compounds used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

A number of targets are useful for the development of highly specific drugs to treat a disease or disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, low complexity, and low cost.

By “fragment” is meant a portion of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain any portion of a polypeptide or nucleic acid molecule or sequence, such as but not limited to, 3, 4, 5, 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. The preparation is at least 75%, at least 90%, and at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least 16 amino acids (e.g., 20 amino acids, 25 amino acids, 35 amino acids, 50 amino acids, 100 amino acids). For nucleic acids, the length of the reference nucleic acid sequence will generally be at least 50 nucleotides (e.g., 60, 75, 100, 200, 300), and in some instances 100 nucleotides or 300 nucleotides or any integer thereabout or therebetween.

By “siRNA” is meant a double stranded RNA. In some instances, an siRNA is 10 or more nucleotides in length (e.g., 18, 19, 20, 21, 22, 23 or 24 nucleotides in length) and has a 2 base overhang at its 3′ end. These dsRNAs can be introduced to an individual cell or to a whole animal; for example, they may be introduced systemically via the bloodstream. Such siRNAs are used to downregulate mRNA levels or promoter activity.

By “specifically binds” is meant a compound or antibody that recognizes and binds a polypeptide of the invention, but which does not substantially recognize and bind other molecules in a sample, for example, a biological sample, which naturally includes a polypeptide of the invention.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than 750 mM NaCl and 75 mM trisodium citrate, less than 500 mM NaCl and 50 mM trisodium citrate, and less than 250 mM NaCl and 25 mM trisodium citrate. Low stringency hybridization can be obtained in the absence of organic solvent, e.g., formamide, while high stringency hybridization can be obtained in the presence of at least 35% formamide, and at least 50% formamide. Stringent temperature conditions will ordinarily include temperatures of at least 30° C., of at least 37° C., and of at least 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In one embodiment, hybridization will occur at 30° C. in 750 mM NaCl, 75 mM trisodium citrate, and 1% SDS. In one embodiment, hybridization will occur at 37° C. in 500 mM NaCl, 50 mM trisodium citrate, 1% SDS, 35% formamide, and 100·mu·g/ml denatured salmon sperm DNA (ssDNA). In another embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than 30 mM NaCl and 3 mM trisodium citrate, and less than 15 mM NaCl and 1.5 mM trisodium citrate. Stringent temperature conditions for the wash steps will ordinarily include a temperature of at least 25° C., of at least 42° C., and of at least 68° C. In one embodiment, wash steps will occur at 25° C. in 30 mM NaCl, 3 mM trisodium citrate, and 0.1% SDS. In another embodiment, wash steps will occur at 42 C in 15 mM NaCl, 1.5 mM trisodium citrate, and 0.1% SDS. In yet a further embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Such a sequence is at least 60% (e.g., 75%, 80%, 85%, 90%, 95%, 97%, 99%) identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

As used herein, the term “subject” refers to any organism to which a composition and/or compound in accordance with the disclosure may be administered, e.g., for experimental, diagnostic, prophylactic, and/or therapeutic purposes. By “subject” is meant a mammal, including, but not limited to, a human or non-human mammal, such as murine, bovine, equine, canine, ovine, or feline. A subject in need thereof is typically a subject for whom it is desirable to treat a disease, disorder, or condition as described herein. For example, a subject in need thereof may seek or be in need of treatment, require treatment, be receiving treatment, may be receiving treatment in the future, or a human or animal that is under care by a trained professional for a particular disease, disorder, or condition.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition. The phrase “preventing an aging or age-related disease or disorder” means reducing the incidences, delaying, or reversing the progression of and/or diseases related to aging or age-related disorders.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, number values such as those in ranges are understood to be within a range of normal tolerance in the art, for example within 2 standard deviations of the mean. These number values can be understood as being within 10%, 9%, 8%, 7%, 6%, 5%, 4%, 3%, 2%, 1%, 0.5%, 0.1%, 0.05%, or 0.01% of the stated value. The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 illustrates sample of nuclei in non-senescent PTEC (left) and in senescent PTECs (right) with DAPI (blue), Ki-67 (green), and γH2AX (red) stains overlaid. Nuclei are shown by the blue dye, proliferation protein marker by Ki-67, and histone protein marker of broken DNA by γH2AX. Original magnification×400.

FIG. 2 shows side-by-side images of the same region under a Senescence-associated beta-galactosidase (SA-β-gal) stain (left) showing senescent PTEC and a DAPI stain (right) of the same region showing PTEC nuclei detected at pH 6. This region has low senescence since there is a very small amount of turquoise in the SA-β-gal image compared to the number of nuclei in the DAPI stain. Original magnification×200. To measure senescence, the total blue area in the SA-β-gal image was divided by the number of nuclei found in the DAPI stain.

FIG. 3 provides a graph demonstrating changes in amount of Ki-67 and γH2AX between PTEC at day 9 without irradiation and day 16 after irradiation. At each of Days 9 and 16, the left column represents percent of cells with Ki-67 (non-senescent) and the right column represents percent of cells with only γH2AX (senescent). Both changes are statistically significant. Individual data are shown by overlaid cross marks. Between Day 9 and Day 16, the number of Ki-67-stained PTECs decreased somewhat, while that of γH2AX-stained cells increased significantly.

FIG. 4 illustrates non-senescent (left) and senescent (right) PTECs in the yolk sacs of zebrafish larvae. Cells were stained with eFluor 450+NucBlue and imaged on the day of injection (day 2 post fertilization). Original magnification×100.

FIGS. 5A-5B present survival curves showing that larvae injected with senescent cells have poorer survival rates than larvae injected with non-senescent cells. At Day 5, the curves from the highest percentage of surviving fish to the lowest percentage are as follows: Control, no injection; Control, Dye injection; Non-senescent (ns) PTECs; Senescent (sen) PTECs.

FIG. 6 provides a flowchart of the cell xenotransplantation protocol.

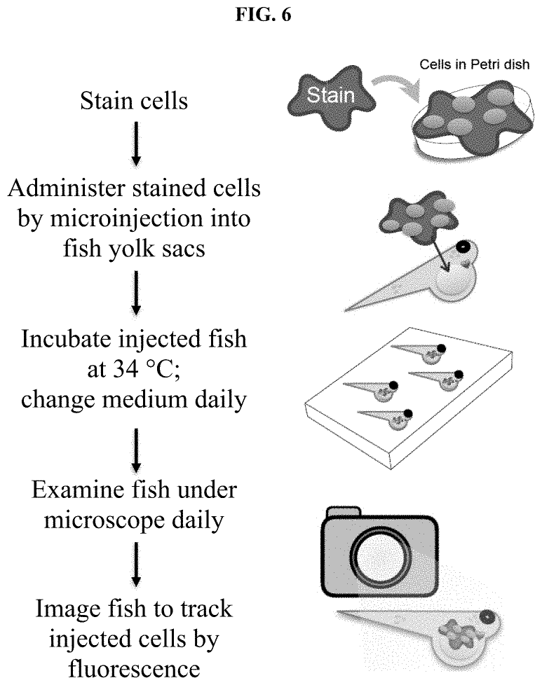

### DETAILED DESCRIPTION OF THE INVENTION

The invention features methods and systems that are useful for senolytic or senotherapeutic drug discovery and examining cellular mechanisms of senescence and the senescence-associated secretory phenotype (SASP). The system may comprise a mammalian cell model for testing senescence-directed cellular strategies. One method of the invention includes a method of analyzing cells in the mammalian cell model system. Another method provides a cost-effective, as well as time-effective, high-throughput method of screening potential candidate substances for testing for decreasing cellular senescence.

### Model System

One embodiment of the invention provides an embryonic zebrafish cell xenograft assay for studying mammalian cell senescence and senescence-directed interventions. This system provides a tool to study the biology of senescent mammalian cells and different forms of senescence-directed interventions (e.g. senolysis) in a preclinical model of a simple and robust organism, which is ideal for high-throughput and pipeline acceleration. Approaches that allow a high-throughput and cell focused exploration of senescence-related biological processes are desired. Using transparent fish, e.g., zebrafish, medaka, as a simple model organism is a promising strategy to achieve this goal.

In an embodiment of the invention, Zebrafish (Danio rerio) may be a useful model organism because their larvae develop outside the body of the adults, they produce hundreds of eggs at a time, their larvae develop extremely quickly, and the larvae as well as some strains of adults are transparent, allowing for easy visualization. Larvae can live for seven days in a single well of a standard 96- or 386-well microtiter plate with the proper nutrients. However, any similar transparent model organism may be used. A model of the effects of Senescence-Associated Secretory Phenotype (SASP) and senescent cells on the body may utilize any transparent or translucent organism similar to fish larvae, for example, but not limited to, zebrafish (Danio rerio) and medaka (Oryzias latipes). To expose the fish to senescence, the yolk sacs of fish larvae may be administered by injection with senescent and/or non-senescent mammalian cells (e.g., human, murine).

In some embodiments, the mammalian cells are stained with one or more dyes. The stains, dyes, and/or labels of the invention include those that identify broken DNA, proliferation, and cellular senescence, or any other similar marker. The larvae may be administered by injection with these cells to determine how SASP affects larval development. Mammalian cells (e.g., epithelial, endothelial, primary tubular epithelial cells (PTECs), human umbilical vein endothelial cells (HUVECs)) may first be cultured, stained, and then injected into a yolk sac of the model fish system (e.g., zebrafish, medaka). The staining techniques of the mammalian cells (e.g., senescent, non-senescent) are optimized and in an amount sufficient for visualizing the cells after they have been injected into the fish model. The stained cells observed in the fish model system may be tracked over time. In one embodiment, the stained cells in the transparent or translucent fish model may be observed and analyzed by fluorescence. SA-βgal staining may be visualized and photographed under, for example, phase contrast and bright field microscopy. Stains for proliferating cells, nuclei, or other components that suggest either senescence, cell cycle arrest, non-senescence, or proliferation (e.g., EdU labeling, Ki-67, DAPI, immunostaining of p16INK4a) may be visualized and photographed by, for example, fluorescence microscopy. The model system may demonstrate that senescent mammalian cells disturb the model embryonic development and lead to a significantly increased death rate as compared to non-senescent mammalian cells from the same source.

Another embodiment may be directed to a zebrafish larvae model system containing mammalian cells (previously transplanted into the yolk sac of the zebrafish larvae) for screening candidate senescence-antagonizing substances, where the mammalian cells have stain or detectable markers for, e.g., senescence, proliferation, nuclei, cell cycle arrest, and the like. Diluting a candidate substance in the surrounding water or medium in which the zebrafish larvae are cultured, allows the candidate substance to be absorbed through the skin and gills of the zebrafish larvae. Alternatively, water-insoluble candidate substances or highly hydrophobic compounds, large molecules, and proteins may be directly injected into the yolk sac, the sinus venosus, or the circulatory system of the zebrafish. The zebrafish may be analyzed in order to visualize the stain or detectable markers, thereby identifying and quantifying cellular senescence or lack thereof.

### Methods

Some embodiments of the invention may provide a method of analyzing cells for senescent and/or non-senescent cells in a system as described here. The method of analyzing cells may comprise:

staining non-senescent and/or senescent mammalian cells (e.g., epithelial, endothelial, PTECs, HUVECs, neuronal) with a detectable marker (e.g., γH2AX, Ki-67, SA-β-gal, DAPI; transgenic expression of fluorescent proteins (e.g., EGFP, dsRed), detectable in vivo cell label or live cell labeling reagents (e.g., fluorescent cell labels e.g., eFluor and NucBlue stains), or the like); introducing one or more of the stained cells (e.g., 10, 20, 50, 75, 100, 150, 200, 250, 300), such as 100-200 cells, into a yolk sac of a fish (e.g., transparent/translucent fish or fish larvae, such as, but not limited to, zebrafish and medaka); incubating the fish or fish larvae in a medium (e.g., water, Phosphate Buffered Saline (PBS), 0.5×E2 embryo medium); and analyzing the fish for senescent and/or non-senescent cells. The stain or marker may include fluorescent cell markers, which differentiate between the transplanted or injected stained cells and those of the zebrafish larvae.

The mammalian cells may be purchased or obtained directly from a mammalian subject. Prior to administering the cells to the zebrafish yolk sac, the mammalian cells may be preconditioned by incubating at a temperature less than that which is ideal for the mammalian cells and more than that which is ideal for zebrafish larvae. For example, the mammalian cells may be maintained at an inclusive temperature of 33° C. to 35° C., e.g., 33° C. to 34° C., for several hours to 24 hours prior to staining, such as with a fluorescent dye or marker. Non-limiting examples of useful stains or markers of cellular senescence, proliferation, cell cycle arrest, damaged DNA, nuclei, and the like, include γH2AX, Ki-67, SA-β-gal, and DAPI.

The stained cells or those containing at least one detectable marker may be introduced into the yolk sac of a transparent or translucent fish by, for example, injection. Injecting the cells may occur by microinjecting the cells into the yolk sac using, for example, a glass capillary needle, injection syringe, injection pipette, or the like. In one embodiment, the injecting step occurs at least 1 day (e.g., 2 days, 3 days) after or 20 or fewer days (e.g., 16, 14, 12, 10, 8, 6, 4, 2) post fertilization. The fish containing the injected cells may be maintained or cultured at a temperature sufficient to sustain the fish and the injected mammalian cells, for example, in an inclusive range of 28° C. to 37° C., such as 34° C. in a medium (e.g., fish water, Phosphate Buffered Saline (PBS), 0.5×E2 embryo medium) conducive to optimally maintaining the fish and injected cells. See, e.g., Niisslein-Volhard, Christiane & Dahm, Ralf. (2001). Zebrafish: A Practical Approach for teaching zebrafish maintenance, culturing, labeling, transplanting, and the like, which is incorporated herein by reference in its entirety. In order to analyze the fish, they may be examined visually, for example, under a microscope.

Another embodiment provides for a method of screening potential candidate substances, such as, but not limited to, senescence-antagonizing drugs using the model system described here. The screening method comprises staining cells, such as non-senescent or senescent mammalian cells (e.g., human, murine, PTECs, HUVECs) with a detectable marker which allows tracking of cells in a recipient fish. Non-limiting examples of detectable markers include γH2AX, Ki-67, SA-β-gal, DAPI; transgenic expression of fluorescent proteins (e.g., EGFP, dsRed), live cell labeling reagents (e.g., fluorescent cell labels e.g., eFluor and NucBlue stains); one that identifies broken or damaged DNA, proliferation, cellular senescence, or the like. The screening method after staining cells further comprises introducing the stained cells into a yolk sac of a fish (e.g., transparent or translucent fish, such as zebrafish, medaka); incubating the fish in a medium; administering a potential candidate substance, where administration may be by, for example, solubilizing the drug in the fish medium or directly injecting the drug into the fish, where the drug may be in a carrier, diluent, or excipient; and analyzing the fish after administration of the candidate substance, more specifically, analyzing the transplanted stained mammalian cells in the fish. The candidate senescence-antagonizing drug may be administered to the fish by dissolving the candidate substance in the fish medium, where the medium may be any solution sufficient for maintaining the fish, such as but not limited to, fish water, Phosphate Buffered Saline (PBS), and 0.5×E2 embryo medium. An alternative method of administrating a candidate substance may occur by directly injecting the candidate substance into the fish.

In one embodiment, the candidate substance may be any that reverses senescent cells to non-senescent cells, prevents the progression of non-senescent cells to senescent cells, induces senescent cell death, suppresses senescent phenotypes without cell killing, induces toxicity to proliferating cells, induces toxicity to senescent cells, suppresses cell senescence, increases in cell senescence, and increases in proliferation, or the like. Non-limiting examples of potential candidate substances may include senotherapeutics, geroprotectors (e.g., melatonin, carnosine, metformin, nicotinamide mononucleotide, delta sleep-inducing peptide), SASP inhibitors, senolytics (e.g., FOXO4-related peptides, inhibitors of bcl-2 family of anti-apoptotic proteins, inhibitors of Ubiquitin-specific processing protease 7 (USP7), senescence-specific killing compound 1 (SSK1), 2-(3,4-dihydroxyphenyl)-3,7-dihydroxychromen-4-one (Fisetin), 4-(4-{[2-(4-Chlorophenyl)-5,5-dimethyl-1-cyclohexen-1-yl]methyl}-1-piperazinyl)-N-[(4-{[(2R)-4-(4-morpholinyl)-1(phenylsulfanyl)-2-butanyl]amino}-3-[(trifluoromethyl)sulfonyl]phenyl) sulfonyl]benzamide (Navitoclax), 1-[(2E)-3-(3,4,5-Trimethoxyphenyl)prop-2-enoyl]-5,6-dihydropyridin-2(1H)-one (Piperlongumine)), and senomorphics (e.g., free radical scavengers and inhibitors of IκB kinase (IKK), nuclear factor kappa B (NF-κB), and the Janus kinase (JAK) pathways). Additional geroprotectors for use in the invention described herein may be found in a curated database of geroprotectors (https://geroprotectors.org/). Alternatively, in other embodiments, where it is desirable to stem or reduce excessive, uncontrolled, or abnormal proliferation of cells, such as for example, in cancer, atherosclerosis, rheumatoid arthritis, psoriasis, idiopathic pulmonary fibrosis, scleroderma, and cirrhosis of the liver, the candidate substance may be an inhibitor of proliferation.

Some embodiments may be directed to the analyzing step, which may comprise quantifying senescence. The mammalian cells may be stained with an enzyme used as a biomarker of cellular senescence, which may be detected at pH 6, such as, for example, SA-β-Gal. The cells may also be stained for nuclei in cells using, for example, DAPI (4′,6-diamido-2-phenylindole), a blue-fluorescent dye. Quantifying senescence may be calculated by dividing the total area of cellular senescence by the number of nuclei in the same area.

A further embodiment provides for screening methods for candidate substances that reverse senescent cells to non-senescent cells, prevent progression of non-senescent cells to senescent cells, result in senescent cell death, suppress senescent phenotypes without cell killing, result in toxicity to proliferating cells, result in toxicity to senescent cells, suppress cell senescence, increase in cell senescence, and increase in proliferation. The screening method described here may be a high-throughput, low cost method of measuring senescence. Any candidate substance found to antagonize senescence may be useful in a treatment and/or prevention of an aging or age-related disease or disorder as determined by visualization and/or quantification of senescence.

In yet another embodiment, a method of treating a subject suffering from an aging or age-related disease or disorder may be personalized by screening candidate substances against the subject's own cells, e.g., human primary cells from the subject that are transplanted into the model system described here. Quantifying a decrease in senescent cells or observing a decrease or reversal of senescent cells to non-senescent cells in the model system with administration of the candidate substance, thereby provides a personalized approach to therapeutic medicine.

A further embodiment may be directed to a preparation of a medicament for the treatment of or protection against aging or age-related diseases or disorders, or symptoms thereof.

Any of the embodiments to methods described here may be in a high-throughput or automated format. In some embodiments, the method of analyzing and method of screening described here may be a high-throughput format, providing a rapid and time- and cost-efficient process for analyzing cells for cellular senescence and identifying candidate senescence-antagonizing substances. The analyzing step reveals, identifies, and/or distinguishes senescent and non-senescent mammalian cells in the system described here. Imaging the cells in the fish yolk sac allow for the detection of the stained cells. See e.g., Pringle et al. 2019, which is incorporated herein by reference in its entirety for its disclosure regarding, for example, zebrafish, cell lines, staining cells, zebrafish xenotransplantation, and imaging. The effects of candidate substances on cellular senescence may be observed and calculated. Non-limiting candidate substances may include senotherapeutics, geroprotectors, which prevent or reverse the senescent state by preventing triggers of cellular senescence, such as DNA damage, oxidative stress, proteotoxic stress, telomere shortening (i.e., telomerase activators); SASP inhibitors, which interfere with pro-inflammatory Senescence Associated Secretory Phenotype (SASP) production, glucocorticoids as potent suppressors of selected components of SASP; statins, including simvastatin, that reduces the expression of pro-inflammatory cytokines (IL-6, IL-8, and MCP-1); JAK1/2 inhibitors, such as ruxolitinib; NF-κB and p38 inhibitors; IL-1α blockers; mitochondrial depleters (e.g., mitophagy); senolytics, which specifically induce cell death in senescent cells, targeting survival pathways, and anti-apoptotic mechanisms, antibodies and antibody-mediated drug delivery medications; and senomorphics, which suppress senescent phenotypes without cell killing.

The therapeutic methods of the invention (which include prophylactic treatment) in general comprise administration of a therapeutically effective amount of the candidate substance identified and described herein, to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, family history, and the like). The compounds herein may be also used in the treatment of any other disorders in which aging or age-related diseases or disorders, or in other embodiments, excessive or abnormal proliferation, may be implicated.

In one embodiment, the invention provides a method of monitoring treatment progress. The method includes the step of determining a level of a diagnostic marker, such as one for cellular senescence (e.g., any target delineated herein modulated by a candidate substance herein, a protein or indicator thereof, etc.) or diagnostic measurement (e.g., screen, assay, quantification of cellular senescence) for identifying, for example, a reversal of senescent cells to non-senescent cells, prevention of progression of non-senescent cells to senescent cells, senescent cell death, suppression of senescent phenotypes without cell killing, toxicity to proliferating cells, toxicity to senescent cells, suppression of cell senescence, increase in cell senescence, or increase in proliferation, in a subject suffering from or susceptible to a disorder or symptoms thereof associated with aging, age-related disease or condition, or diseases or conditions of excessive or abnormal proliferation, in which the subject has been administered a therapeutic amount of a candidate substance (e.g., senotherapeutics, geroprotectors, Senescence Associated Secretory Phenotype (SASP) inhibitors, senolytics, and senomorphics) described herein sufficient to treat the disease or symptoms thereof. The level of cellular senescence determined in the method can be compared to control in either healthy normal controls or in other afflicted patients to establish the subject's disease status. In some embodiments, a second level of marker in the subject may be determined at a time point later than the determination of the first level, and the two levels may be compared to monitor the course of disease or the efficacy of the therapy. In certain embodiments, a pre-treatment level of a marker in the subject is determined prior to beginning treatment according to this invention; this pre-treatment level of a marker can then be compared to the level of the marker in the subject after the treatment commences, to determine the efficacy of the treatment.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what may be regarded as the invention.

### EXAMPLES

### Example 1: Method for Injecting Senescent Cells from Other Organisms into Zebrafish

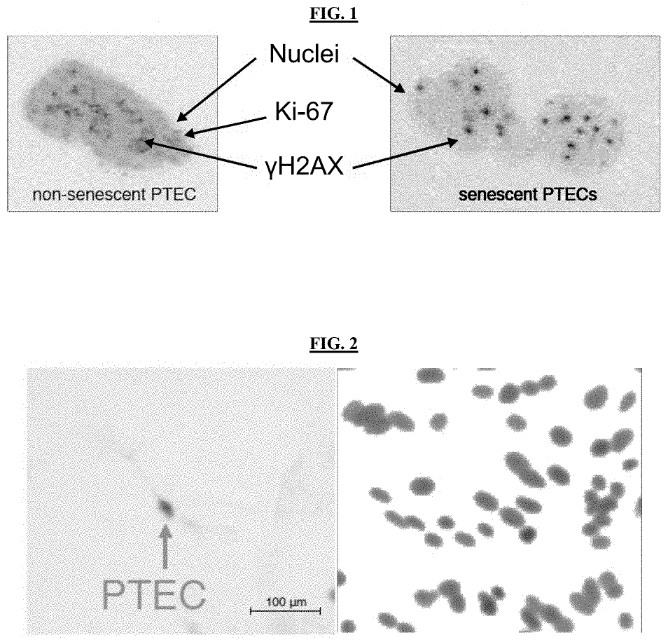

Cells from multiple mouse strains that were transplanted into zebrafish were imaged, including some cells on day 9 after they were procured from the mouse as a non-senescent group and a second group that were irradiated with gamma radiation (10 Gy) on day 7 for imaging on day 16. The second group became senescent. Two different methods of quantifying senescence in PTECs (primary tubular epithelial cells) were automated using ImageJ's built-in scripting language, IJM. The first was comprised of a double-stain for Ki-67 and γH2AX (FIG. 1). Ki-67 is a protein associated with cellular division and growth, while γH2AX contributes to the repair of broken DNA (Kuilman T, 2010). Senescent cells repair broken DNA, but are themselves no longer growing or dividing, so senescent cells contain just γH2AX. In contrast, non-senescent cells divide and, in the process, repair broken DNA, thus they contain both Ki-67 and γH2AX. They may also contain just Ki-67 or neither if they are not currently growing or dividing. γH2AX presents as red spots in the cell nucleus when stained, while Ki-67 is an intranuclear diffuse green cloud. The presence of 5 or more spots of γH2AX coupled with the absence of Ki-67 indicated a senescent cell. Cells containing both Ki-67 and γH2AX and cells containing just Ki-67 were indicative of non-senescent cells. The process of identifying all of the cell nuclei through a DAPI stain, then identifying and removing all the cells containing Ki-67 was automated. The process further counted all remaining cells with 5 or more spots of γH2AX. As a result, a count of the total number of cells and the number of cells identified as senescent were counted.

### Example 2: Cell Quantification

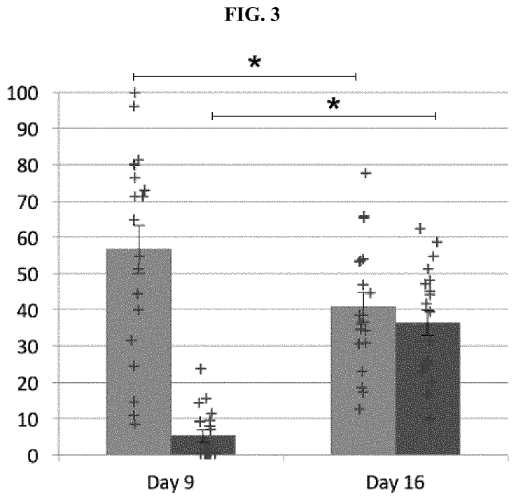

Another method of quantifying cells stained with SA-β-gal (senescence-associated beta-galactosidase) was performed by first imaging one SA-β-gal stain at pH 6 and one DAPI stain (FIG. 2). Using the Feulgren-Light Green color deconvolution described in Ruifrok 2001 and implemented by Landini, the total area of SA-β-gal was calculated, based on a threshold for the Feulgren color which appeared as turquoise. The total area was divided by the number of cell nuclei in the DAPI stained image as a rough measure of senescence per cell.

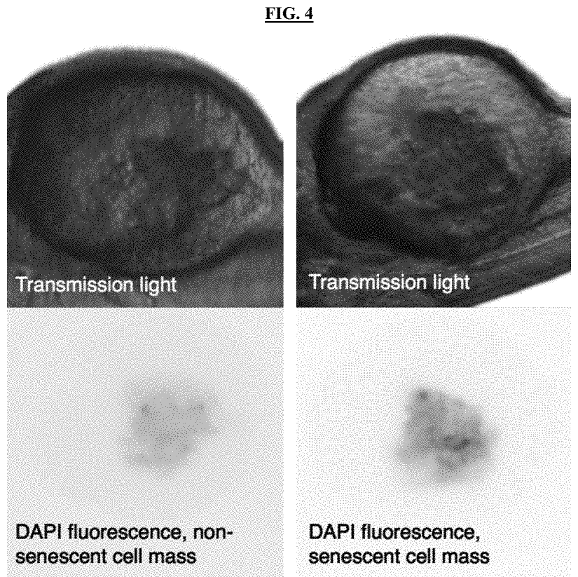

Senescence as measured by the double staining protocol increased from day 9 to day 16. The percentages of Ki-67 cells representing the non-senescent cell groups underscore the real value as some non-senescent cells may not contain any Ki-67 or γH2AX. A senescent cell was Ki-67 negative and γH2AX positive, while a non-senescent cell contained both, just Ki-67, or neither. To filter out dead cell nuclei, cells without Ki-67 or γH2AX were not included in the total count. Non-senescent cells without Ki-67 were constant between the populations and so would not affect the difference between the two groups, i.e., non-senescent and senescent. The results from the analysis of the double-stained PTECs are shown in a chart. FIG. 3 illustrates the average percentage of cells per image containing Ki-67 and the average percentage of cells per image with 5 or more γH2AX dots and no Ki-67. A t-test produced p-values of 0.026 for the Ki-67 data and 1.33×10⁻⁹ for the γH2AX data, so both were considered statistically significant.

### Example 3: Cell Transplantation

For cell transplantation, MDIBL's Nacre/Nacre and Flk/Nacre zebrafish lines were used because of their translucency and hardiness. Eggs were collected on day 0, dechorionated on day 1 or day 2, and injected cells on day 2. Microinjectors with glass capillaries were used to inject cells into the fish larval yolk sacs. The larvae were anesthetized with tricaine as needed. In order to track the injected cells, eFluor and NucBlue stains were used. PTECs were produced from the kidneys of male C57Bl/6 mice. Typically, 30 larvae were used to begin with for each of an uninjected control group and a dye injected control, and with up to 100 larvae for the senescent and non-senescent dyed cell injected groups. Looking under a fluorescence microscope, each larva was examined and the ones with the best injections were selected.

Although individual cells could not be seen at the magnification used, the size and brightness of the cell mass, as well as any malformations in the larvae that had been injected and any leakage of cells from the yolk sac were determined. The best injected fish from each group were selected, rinsed to remove the tricaine, and one fish per well in 96-well plates were placed with 200 μL of PBS medium in each well. The plates were then incubated at 34° C. for a period of time sufficient for the marker to be absorbed by the cells, which may be, for example, at least 5 minutes (e.g., 10 minutes, 15 minutes).

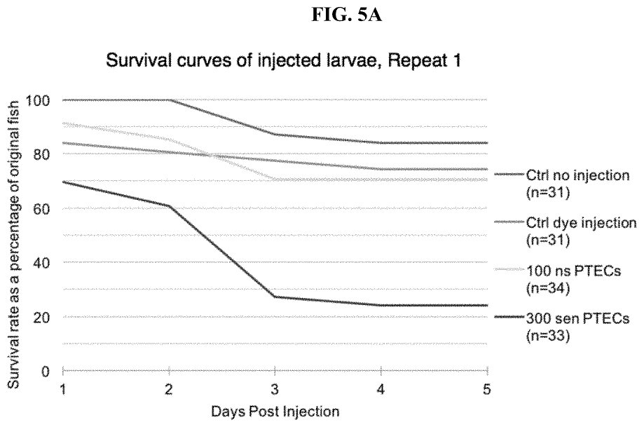

Although the fish may be maintained at 28° C. and the stained mammalian cells may be cultured in temperatures closer to body temperature (e.g., 37° C.), 34° C. was determined to be sufficient to successfully maintain both. Each day the fish were monitored, any dead or malformed larvae were removed and their medium exchanged daily. Representative fish were imaged as desired. Cell masses inside the yolk sacs of zebrafish were imaged. Non-senescent cell masses were found to be fainter than senescent cell masses, which may be because the non-senescent cells are smaller and do not stain as well as the senescent cells. FIG. 4 shows larval yolk sacs immediately post-injection under transmission and DAPI fluorescent light, showing the differences between successful non-senescent and senescent cell injections.

### Example 4: In Vivo Animal Model

Zebrafish (Danio rerio) fertilized eggs were collected and maintained at 28.5° C. At 48 h post fertilization (hpf), embryos hatched from the chorion or were dechorionated to become free-living larvae. Thereafter, mammalian cells of interest (e.g. murine, human, or other species) which had been made senescent by different stress models (e.g., gamma-irradiation, bleomycin, mitochondrial dysfunction associated senescence) were injected into the yolk sac of anesthetized zebrafish larvae (0.003% tricaine). Non-senescent cells served as controls. Using embryos for this approach had the advantage of avoiding the development of an adaptive immune response, which would have rejected the grafted cells. Several research groups have successfully applied xenotransplantation to embryonic zebrafish in the context of cancer research (Barriuso et al, 2015). For tracking purposes, the cells were labeled by cell membrane dyes or by transgenic expression of fluorochromes. Cell injections were performed using borosilicate glass capillary needles and electric microinjectors to transfer 10,000-20,000 cells/μl into the yolk of the embryo. Incorrectly injected embryos, i.e. embryos without cells inside of the yolk or with cells placed outside the yolk, are discarded. Zebrafish embryos are translucent allowing for easy visual cell tracking. During the subsequent 5 day observation period, there were three major readouts: a) survival of zebrafish larvae; b) survival of transplanted cells (demonstrating the efficiency of anti-senescence strategies); and c) disturbance of physiological larval development. For monitoring daily senescent cell survival, fluorescence microscopy with semiquantitative cell counts was employed. Regular microscopy with anatomical assessment was used for monitoring physiological development and organogenesis. To monitor angiogenesis, fish with fluorescent vascular structures (e.g., Tg(flk:mCherry)) were used and examined under fluorescent light. At the end of the observation period, zebrafish embryos were fixed and mounted to take images, growth differences were measured, and injected cells were counted. Alternatively, fish were collected to extract RNA for expression studies by qRT-PCR.

### Example 5: Cell Survival

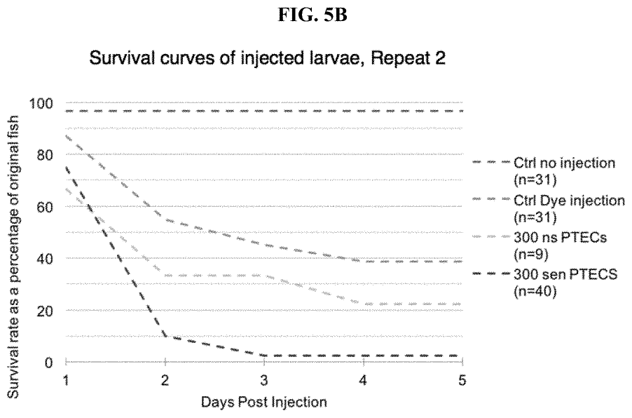

The large-scale effects of senescent PTECs on zebrafish larvae were determined. The cell survival curves illustrated survival rate as a percentage of original fish e over days post-infection for no injection control, dye injection control, non-senescent (ns) PTECs, and senescent (sen) PTECs. Although only the first two experiments are shown in FIGS. 5A and 5B, three repeats of the same experiment were performed with the same result. Dye-injected fish had worse survival rates than un-injected fish, non-senescent cell injected fish performed worse than the dye-injected, while the senescent cell injected fish performed the worst of all groups.

While PTEC cell injections of either kind were found disadvantageous for larvae, senescent cell injections were particularly deleterious, especially in the first two days after injection. The predominant result was death, suggesting a drastic effect in injected larvae. Fish that survived the first two days after injection tended to survive until day 7, perhaps because the PTECs had died off or because of some resistance to the negative effects caused by the injections.

### Example 6: Interventional Procedure

For screening potential candidate substances and testing them for inducing, delaying, or reversing senescence in the model system described here, fish water is supplemented with the respective candidate substance, which may be a senotherapeutic drug compound, for the experimental group and with solvent for the control group. Alternatively, the candidate substance is directly injected with the cells into the yolk sac of the fish.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

### REFERENCES

Childs B G, Li H, van Deursen J M. Senescent cells: a therapeutic target for cardiovascular disease. J Clin Invest. 2018 Apr. 2; 128(4):1217-1228.

Gorgoulis V et al. Cellular Senescence: Defining a Path Forward. Cell. 2019 Oct. 31; 179(4):813-827.

Schmitt R. Senotherapy: growing old and staying young? Pflügers Arch. 2017 September; 469(9):1051-1059.

Barriuso J et al. Zebrafish: a new companion for translational research in oncology. Clin Cancer Res. 2015 Mar. 1; 21(5):969-75.

Kim E C, Kim J R. Senotherapeutics: emerging strategy for healthy aging and age-related disease. BMB Rep. 2019 January; 52(1):47-55.

Avci M E et al. Development of a novel xenograft model in ache mutants using liver cancer cell lines. Scientific Reports 2018; 8:1570. doi: 10.1038/s41598-018-19817-w.

Baker D J et al. Naturally occurring p16(Ink4a)-positive cells shorten healthy lifespan. Nature. 2016 Feb. 11; 530(7589):184-9. doi: 10.1038/nature16932

Kuilman T et al. The essence of senescence. Genes Dev. 2010 Nov. 15; 24(22):2463-79. doi: 10.1101/gad.1971610.

Ruifrok, A C and Johnston, D A. Quantification of histochemical staining by color deconvolution. Anal. Quant. Cytol. Histol. 2001 23: 291-299, PMID 11531144. The color deconvolution plugin implemented by Gabriel Landini, and more information about it, can be found at http://www.mecourse.com/landinig/software/cdeconv/cdeconv.html and https://imagej.net/Colour_Deconvolution.

What is claimed is:

1. A method of analyzing cells, comprising:
staining mammalian cells;
introducing one or more of the stained mammalian cells into a yolk sac of a fish;
incubating the fish in a medium; and
analyzing the fish.

2. The method of claim 1, wherein the introducing step comprises injecting the stained mammalian cells at 2 days post fertilization.

3. The method of claim 1, wherein the incubating step occurs at 34° C.

4. The method of claim 1, wherein the analyzing step comprises examining the fish under a microscope.

5. The method of claim 4, wherein the analyzing step further comprises imaging the fish.

6. The method of claim 1, further comprising administering a candidate substance to the fish.

7. The method of claim 6, wherein administering occurs by adding the candidate substance to the medium.

8. The method of claim 6, wherein the stained cells are senescent cells and/or non-senescent cells.

9. The method of claim 6, wherein the candidate substance is selected from senotherapeutics, geroprotectors, Senescence Associated Secretory Phenotype (SASP) inhibitors, senolytics, and senomorphics.

10. The method of claim 8, wherein analyzing the fish for any one or more of: a reversal of senescent cells to non-senescent cells, prevention of progression of non-senescent cells to senescent cells, senescent cell death, suppression of senescent phenotypes without cell killing, toxicity to proliferating cells, toxicity to senescent cells, suppression of cell senescence, increase in cell senescence, and increase in proliferation.

11. The method of claim 4, wherein the analyzing step comprises quantifying senescence.

12. The method of claim 11, wherein quantifying is by dividing total positive SA-β-Gal-stained area by the number of nuclei in DAPI-stained area.

13. A method of screening for a candidate substance, comprising:
staining mammalian cells;
introducing one or more of the stained mammalian cells into a yolk sac of a fish;
incubating the fish in a medium;
administering a candidate substance; and
analyzing the fish after administration of the candidate substance.

14. The method of claim 13, wherein administering occurs by adding the candidate substance to the medium.

15. The method of claim 13, wherein administering occurs by injecting the candidate substance into the fish.

16. The method of claim 13, wherein the candidate substance is selected from: senotherapeutics, geroprotectors, Senescence Associated Secretory Phenotype (SASP) inhibitors, senolytics, and senomorphics.

17. The method of claim 13, wherein analyzing the fish for any one or more of: a reversal of senescent cells to non-senescent cells, prevention of progression of non-senescent cells to senescent cells, senescent cell death, suppression of senescent phenotypes without cell killing, toxicity to proliferating cells, toxicity to senescent cells, suppression of cell senescence, increase in cell senescence, and increase in proliferation.

18. The method of claim 13, wherein the analyzing step comprises quantifying senescence.

19. The method of claim 13, wherein the cells are stained with SA-β-Gal and DAPI.

20. The method of claim 19, wherein quantifying is calculated by dividing total positive SA-β-Gal-stained area by the number of nuclei in DAPI-stained area.
